(19) United States
(12) Patent Application Publication
(10) Pub. No.: US 2022/0401082 A1
(43) Pub. Date: Dec. 22, 2022

(54) BIOLOGICAL SAMPLE COLLECTION PAD

(71) Applicant: ZYMO RESEARCH CORPORATION, Irvine, CA (US)

(72) Inventors: Stanislav FORMAN, Newport Beach, CA (US); Xi-Yu JIA, Newport Beach, CA (US); Ian FORSYTHE, Irvine, CA (US)

(21) Appl. No.: 17/770,601

(22) PCT Filed: Oct. 20, 2020
(86) PCT No.: PCT/US2020/056426

§ 371 Date: Apr. 20, 2022

Related U.S. Application Data
(60) Provisional application No. 62/923,704, filed on Oct. 21, 2019.

(51) Int. Cl. A61B 10/00

(52) U.S. Cl. CPC A61B 10/0038; B01L 2300/0825; B01L 2300/0816; B01L 2200/141; B01L 2200/0689; B01L 2300/126

(57) ABSTRACT

Aspects of the invention provide a biological sample collection pad (e.g., for the collection of fecal, liquid or tissue samples). Method of using such pads and analyzing collected samples are also provided.

This application claims the benefit of U.S. Provisional Patent Application No. 62/923,704, filed Oct. 21, 2019, the entirety of which is incorporated herein by reference.

### BACKGROUND OF THE INVENTION

### 1. Field of the Invention

The present invention relates generally to the fields of biology and chemistry. More particularly, it concerns biological sample collection pads and methods for the use thereof.

### 2. Description of Related Art

Recently, individualized biological sample analysis and have revolutionized the way in which patient data and health can be analyzed. However, there remain significant challenges in how samples can be effectively collected and stored. To date there remains a need for more effective sample collection devices and methods for theirs use.

### SUMMARY OF THE INVENTION

An exemplary embodiment of the present disclosure provides a sample collection pad comprising: (a) a sample collection strip; and (b) a backing material having a larger surface area than the sample collecting strip and which is water soluble, said backing material comprising at least two layers, (i) a paper layer and (ii) a tissue layer, wherein the tissue layer is positioned between the paper layer and the sample collection strip. In certain aspects, the tissue layer comprises a material that has a higher porosity than the paper layer. In some aspects, the tissue layer has a soft tactile surface. In several aspects, the backing material comprises a starch. In certain aspects, the tissue and paper layers are bound together by a water soluble adhesive. In other aspects, tissue or paper layers comprise a plant-based nonwoven material. In some aspects, the tissue and paper layer conform with INDA/EDANA (Association of the Nonwoven Fabrics Industry/European Disposables and Nonwovens Association) guidelines for flushability. In other aspects, the tissue layer completely dissolves in water in less than 10 minutes. In certain specific aspects, the sample collection strip comprises a water insoluble material, or a porous or fibrous material. In some aspects, the sample collection strip comprises vinyl, polyethylene(PE), polypropylene(PP), or polyethyleneterephthalate(PET). In particular aspects, the sample collection strip comprises a matter that is essentially non-reactive with biological materials. In some aspects, sample collection strip comprises more than one material layer. In several aspects, the collection pad is sterile. In certain aspects, the collection pad is free from nucleic acid material. In other aspects, the collection strip comprises a viral, or microbial, or parasite, or DNA or RNA standard. In some aspects, the sample collection strip comprises an adhesive between the strip and the tissue layer. In some particular aspects, the sample collection strip utilizes an acrylic or silicone-based adhesive. In further aspects, the sample collection strip comprises a biological sample. In additional aspects, the biological sample comprises material from a wound, a saliva sample, a fecal sample, a urine sample, a mucous sample or a vaginal sample. In specific aspects, the biological sample is a fecal sample. In certain aspects, the pad is sealed in a packaging slip or pouch. In some particular aspects, the packaging slip may be air-tight, vacuum sealed, or have a sterile interior. In certain aspects, the backing material is 1, 2, 3, 4, 5, 6, 7, 8, 9 or 10 layers.

In a further embodiment, there is provided a kit comprising a pad accordingly to any one of the embodiments and aspects described herein and a sample storage container. In certain aspects, the sample storage container is a resealable tube. In some aspects, the sample storage container is sterile. In specific aspects, the sample storage container is free of nucleic acid material. In other aspects, the sample storage container comprises a viral, or microbial, or parasite, or DNA or RNA standard. In particular aspects, the sample storage container comprises a stabilization reagent. In further aspects, the stabilization reagent comprises an organic solvent or a chaotropic agent. In another aspect, the stabilization reagent comprises guanidinium thiocyanate. In additional aspects, the kit further comprises instructions, cleaning pads, sterile gloves, secondary shipping containers or envelopes, and/or labeling materials.

Still a further embodiment of the invention provides a method of collecting a biological sample comprising: (a) obtaining a pad according to the present disclosure (including for example, a pad according any one of claims 1-25); and (b) contacting the sample collection strip of the pad to biological material thereby collecting a biological sample. In some aspects, the method additionally comprises separating the sample collection strip from the backing material. In other aspects, the method further comprises transferring the sample collection strip comprising the biological sample to a sample storage container. In further aspects, the method additionally comprises labeling the sample storage container. In some aspects, the method includes placing the sample storage container into a secondary container to prevent leakage during transport or storage. In certain aspects, the method further comprises providing the sample storage container to a third party or mailing the sample storage container. In several aspects, the sample storage container is a resealable tube. In some aspects, the sample storage container is sterile. In specific aspects, the sample storage container is free of nucleic acid material. In other aspects, the sample storage container comprises a viral, or microbial, or parasite, or DNA or RNA standard. In particular aspects, the sample storage container comprises a stabilization reagent. In further aspects, the stabilization reagent comprises an organic solvent or a chaotropic agent. In a specific aspect, the stabilization reagent comprises guanidinium thiocyanate. In still further aspects, the method further comprises disposing of the backing material. In a particular aspect, the backing material is discarded in a toilet.

In yet still further aspects, the biological sample comprises material from a wound, a saliva sample, a fecal sample, a urine sample, a mucous sample or a vaginal sample. In another aspect, the method further comprises isolating biological material from the sample collection strip. In certain aspects, the method additionally comprises isolating DNA and/or RNA from sample collection strip. In some aspects, the method further comprises analyzing biological material from the sample collection strip. In specific aspects, the method comprises analyzing DNA and/or RNA from the sample collection strip. In several aspects, analyzing comprises sequencing, hybridization and/or PCR. In further aspects, the method additionally comprises preparing a report with the results of the analysis. In another aspect, the analyzing comprises identifying constituent microbial contents in the sample. In certain specific aspects, the method comprises identifying viruses, bacteria and/or parasites present in the sample. In particular aspects, the method comprises quantifying the microbial contents of the sample.

In one example (discussed further below), fecal samples were collected from various donors using either an embodiment of the present disclosure, or the conventional method: a small scoop which is used to acquire a consistent quantity from solid stool.

While fecal samples were obtained for the data collected in the example discussed, exemplary embodiments of the present disclosure can be used on any sample type with non-biased representative results (e.g. skin, blood, saliva, tissue, urine, etc.). A sample for use according to embodiments of the present disclosure may be any sample that comprises or potentially comprises a nucleic acid. For example, the sample may comprise genomic DNA, plasmid DNA or RNA. A sample can be obtained from a variety of sources such as from an animal subject, a plant or from a cell line or tissue bank. A sample may be a fresh sample or a frozen or desiccated sample. For example, a sample from an animal may be a blood sample, a urine sample, a fecal sample, a tissue sample (e.g., a biopsy), a saliva sample, or a hair sample.

After sample collection, the samples were processed and analyzed with the ZymoBIOMICS™ Service—Targeted Metagenomic Sequencing (Zymo Research, Irvine, Calif.).

DNA extraction was performed using one of three different DNA extraction kits, depending on the sample type and sample volume. In most cases, the ZymoBIOMICS™ DNA Miniprep Kit (Zymo Research, Irvine, Calif.) was used. For low biomass samples, such as skin swabs, the ZymoBIOMICS™ DNA Microprep Kit (Zymo Research, Irvine, Calif.) was used as it permits for a lower elution volume, resulting in more concentrated DNA samples. For a large sample volume, the ZymoBIOMICS™-96 MagBead DNA Kit (Zymo Research, Irvine, Calif.) was used to extract DNA using an automated platform.

Bacterial 16S ribosomal RNA gene targeted sequencing was performed using the Quick-16S™ NGS Library Preparation Kit (Zymo Research, Irvine, Calif.). The bacterial 16S primers used amplified the V1-V2 or V3-V4 region of the 16S rRNA gene. These primers have been custom-designed by Zymo Research to provide the best coverage of the 16S gene while maintaining high sensitivity. The sequencing library was prepared using an innovative library preparation process in which PCR reactions were performed in real-time PCR machines to control cycles and therefore prevent PCR chimera formation. The final PCR products are quantified with qPCR fluorescence readings and pooled together based on equal molarity. The final pooled library was cleaned up with Select-a-Size DNA Clean & Concentrator™ (Zymo Research, Irvine, Calif.), then quantified with TapeStation® and Qubit®.

The final library was sequenced on Illumina® MiSeg™ with a v3 reagent kit (600 cycles). The sequencing was performed with >10% PhiX spike-in.

Amplicon sequences were inferred from raw reads using the Dada2 pipeline (Callahan et al, 2016). Chimeric sequences were also removed with the Dada2 pipeline. Taxonomy assignment, composition barcharts, alpha-diversity and beta-diversity analyses were performed with Qiime v.1.9.1 (Caporaso et al., 2010). Taxa that have an abundance significantly different among groups were identified by LEfSe (Segata et al., 2011) with default settings if applicable. Other analyses were performed with in-house scripts.

The data obtained in the examples discussed herein demonstrate that embodiments of the present disclosure were successful in collecting fecal samples for microbiomic analysis when compared to the conventional method.

The data demonstrates that both methods of fecal collection yield similar distributions of bacterial phyla. In comparison to a pre-defined fecal standard, the Firmicutes, Bacteroidetes, and the Actinobacteria phyla have the largest representation. The data further demonstrates similar distributions of bacteria at the genus level are observed.

Accordingly, embodiments of the invention described in this disclosure can collect fecal samples for downstream analysis of the microbiome in a reliable manner, without a loss of microbial biodiversity or bias towards particular microbes when compared to the conventional ‘scoop’ method.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a top elevation view of a biological sample collection pad according to an exemplary embodiment of the present disclosure;

FIG. 2 is a front elevation of the example biological sample collection pad of FIG. 1;

FIG. 3 is a rear elevation view of the example biological sample collection pad of FIG. 1;

FIG. 4 is a right side plan view of the example biological sample collection pad of FIG. 1;

FIG. 5 is a left side plan view of the example biological sample collection pad of FIG. 1;

FIG. 6 is a bottom elevation view of the example biological sample collection pad of FIG. 1;

FIG. 7 is a top elevation view of a biological sample collection pad according to an exemplary embodiment of the present disclosure;

FIG. 8 is a front elevation of the example biological sample collection pad of FIG. 7;

FIG. 9 is a rear elevation view of the example biological sample collection pad of FIG. 7;

FIG. 10 is a right side plan view of the example biological sample collection pad of FIG. 7;

FIG. 11 is a left side plan view of the example biological sample collection pad of FIG. 7; and

FIG. 12 is a bottom elevation view of the example biological sample collection pad of FIG. 7;

FIG. 13 displays the microbial composition (phylum) of 25 fecal samples, which were collected using an embodiment of the present disclosure;

FIG. 14 displays the microbial composition (phylum) of fecal samples, which were collected using the conventional method of fecal collection;

FIG. 15 displays the microbial composition (genus) of 25 fecal samples, which were collected using an embodiment of the present disclosure;

FIG. 16 displays the microbial composition (genus) of fecal samples, which were collected using the conventional method of fecal collection;

FIG. 17 displays the microbial composition (phylum) of 10 replicates of the same fecal standard; and

FIG. 18 is a top elevation view of a biological sample collection pad according to an exemplary embodiment of the present disclosure.

### DESCRIPTION OF ILLUSTRATIVE EMBODIMENTS

### I. Illustrative Embodiments

In certain aspects, exemplary embodiments of the invention provide a sample collection pad. Preferably, the pad comprises a collection matrix with a backing material. In some aspects the backing material comprises 1, 2, 3, 4, 5, 6, 7, 8, 9 or 10 layers. In some aspects, the backing material is a water soluble material. For example, the backing material can be a starch-based material. In specific aspects, the sample collection pad is used to collect fecal samples. The sample collection matrix is then sealed for analysis, while the backing materials can be disposed of in the toilet by virtue of its water solubility.

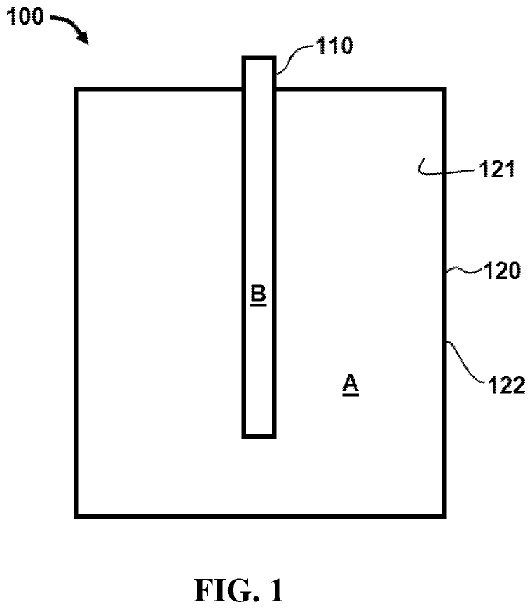

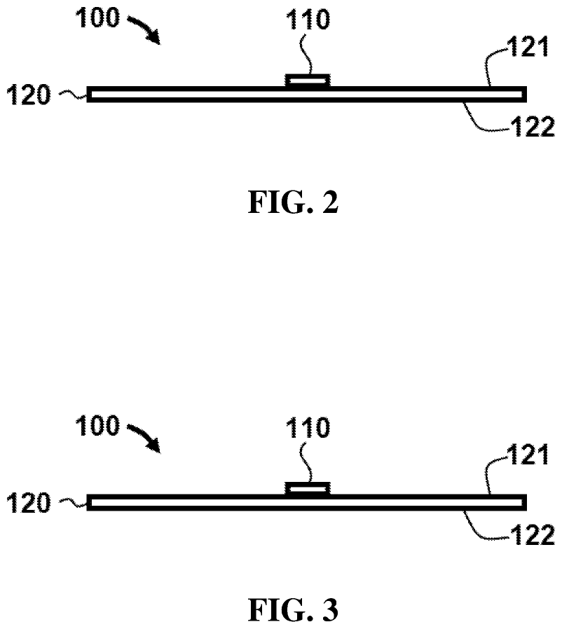

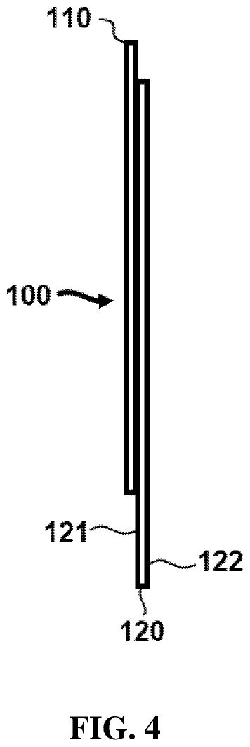

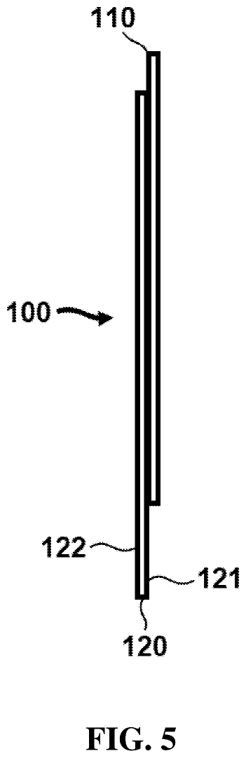

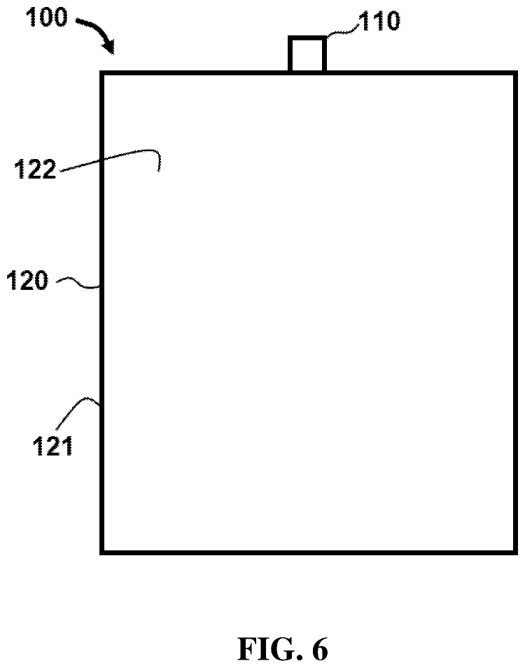

Referring initially to FIGS. 1-6 an embodiment of a biological sample collection pad 100 is shown in front, rear, side, top and bottom views. In the embodiment shown, biological sample collection pad 100 comprises a sample collection strip 110 and a backing material 120. As shown in FIG. 1, backing material 120 has a surface area “A” defined by the outer edge boundaries of backing material 120. In addition, collection strip 110 has a surface area “B” that is defined by the outer edge boundaries of collection strip 110. In the embodiment shown, surface area “A” of backing material 120 is larger than surface area “B” of collection strip 110.

In the embodiment shown in FIGS. 1-6, backing material 120 comprises a first layer 121 and a second layer 122, where first layer 121 is positioned between second layer 122 and collection strip 110. In other embodiments, backing material 120 may comprise only one layer of material. In still other embodiments, backing material 120 may comprise more than two layers. In specific embodiments, first layer 121 is a tissue layer and second layer 122 is a paper layer, so that a tissue layer is positioned between a paper layer and collection strip 110. In certain embodiments, the tissue layer is bonded to the paper layer by thermosealing.

In the embodiment shown in FIGS. 1-6, collection strip 110 is positioned with respect to the backing material 120 such that the top portion of collection strip 110 extends beyond the top portion of backing material 120, while the bottom portion of collection strip 110 does not extend to the bottom portion of backing material 120. It is understood that the terms “top” and “bottom” are only used for reference to the figures, and not intended to designate any directional aspect of the embodiment, including e.g. during use.

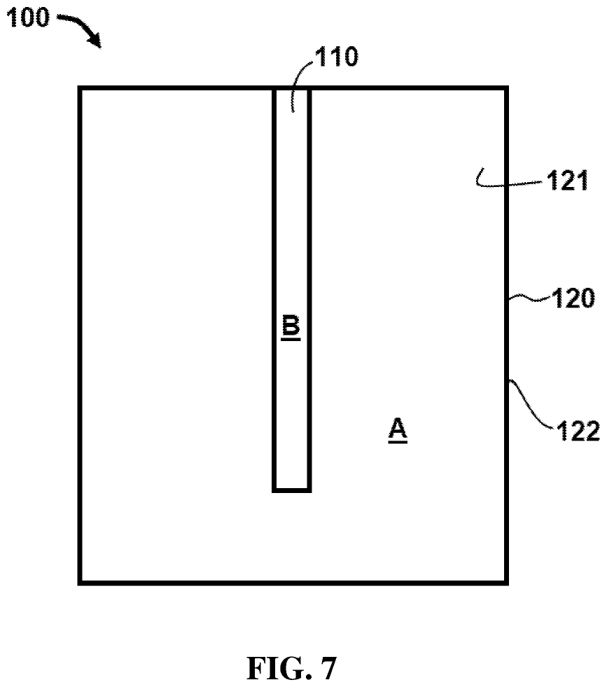

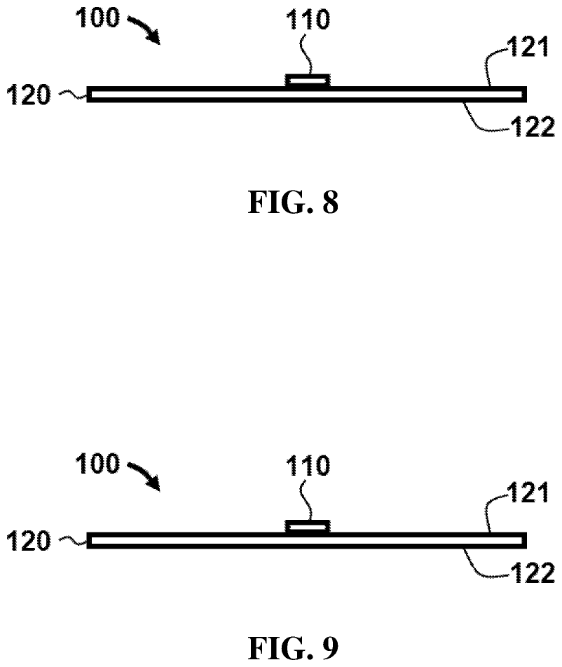

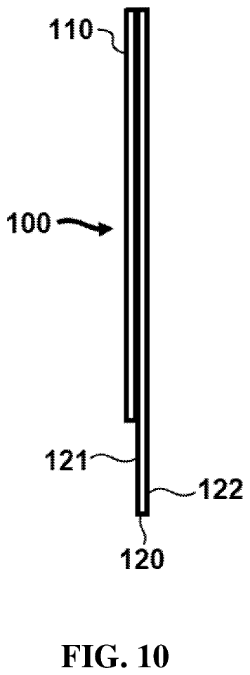

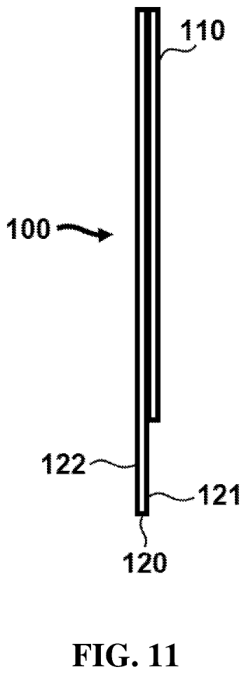

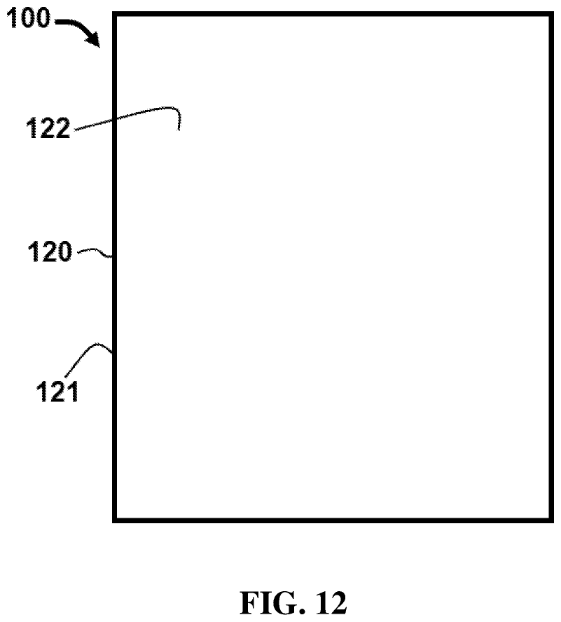

In other embodiments, collection strip 110 may be positioned in a different configuration with respect to backing material 120. For example, referring now to FIGS. 7-12, biological sample collection pad 100 is configured such that collection strip 110 does not extend beyond the top portion of backing material 120. Other aspects of the embodiment shown in FIGS. 7-12 are equivalent to those of the embodiment shown in FIGS. 1-6. For example, in the embodiment shown in FIGS. 7-12 backing material 120 comprises first layer 121 and second layer 122, with first layer 121 positioned between second layer 122 and collection strip 110. First layer 121 can be a tissue layer and second layer 122 can be a paper layer in the embodiment shown in FIGS. 7-12.

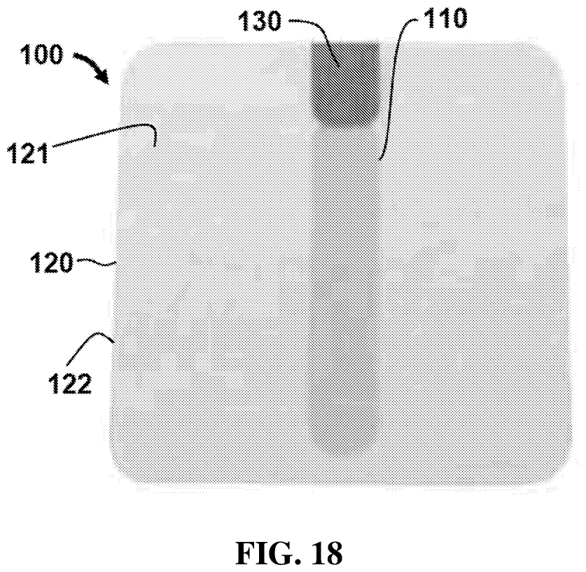

FIG. 18 illustrates an alternate embodiment of sample collection pad 100 with an upper tab 130 (e.g. the shaded portion shown in the figure) for grasping for removing the strip 110.

The layers may comprise one or more materials. For example, in some embodiments the tissue or paper layers can comprise sprayed paper fiber, dissolvable paper, dissolvable cellulose, papier mache, degradable pulp board, dissolvable fabric, nonwoven materials, starch materials, and sheets created from the spraying of paper pulp onto a wire mesh shape or combinations thereof. In specific embodiments, the tissue or paper layer may comprise water soluble paper comprising sodium carboxymethyl cellulose and wood pulp (e.g. such as those available from SmartSolve® Industries, US).

In other embodiments, the tissue or paper layer may comprise a starch-based material, and in a specific embodiment, is a starch-based material. In some embodiments, the starch-based material is preferably natural, derived from plants or other organic materials. In particular embodiments, the starch material is derived from a plant selected from the group consisting of potato, wheat, corn, and rice. Such starch-based materials are now generally known and include those disclosed in U.S. Pat. Nos. 4,863,655, 5,266,368, and 6,054,204, which are incorporated by reference in their entirety. Known useful starch-based material, include but are not limited to Renature® (STOROpack, Germany), ECO-FOAM® (National Starch & Chemical, UK) and others such as Beauty Biofoam disclosed in JP5430052B2, incorporated herein by reference.

In other embodiments, the tissue or paper layer may comprise nonwoven fiber material. To enable biodegradability the fibers are preferably cellulosic in certain embodiments. The cellulosic fibers may for example be natural cellulose fibers such as wood pulp fibers, or manufactured cellulose fibers, such as rayon fibers including viscose rayon. Preferred cellulose fibers include, but are not limited to, digested fibers, such as kraft, pre-hydrolyzed kraft, soda, sulfite, chemi-thermal mechanical, and thermo-mechanical treated fibers, derived from softwood, hardwood or cotton linters. Other example cellulose fibers include, but are not limited to, kraft digested fibers, including pre-hydrolyzed kraft digested fibers. Non-limiting examples of cellulosic fibers suitable for use in this invention are the cellulose fibers derived from softwoods, such as pines, firs, and spruces. Other suitable cellulose fibers include, but are not limited to, those derived from Esparto grass, bagasse, kemp, flax, hemp, kenaf, and other lignaceous and cellulosic fiber sources.

In some embodiments, the tissue and paper layer are bonded by a water soluble binding agent. In specific embodiments the tissue and paper layer are bonded by a polyvinyl alcohol (PVA) resin. In specific embodiments, the tissue and paper layer are bonded by thermosealing the layers together to activate the binding agents described above. In other embodiments, the layers are bonded by a pressure sensitive adhesive or via ultrasonic welding.

In certain embodiments, the backing material is cut in a 4″×4″ square to accommodate the collection of fecal samples. In other embodiments, the backing material may be cut into different sizes and shapes to accommodate the convenient collection from other sample sources.

In certain embodiments, the backing materials comprises tissue and paper layers that disperse in stationary water within about one (1) minute, or about 2 minutes, or about 3 minutes, or about 4 minutes, or about 5 minutes to about 10 minutes, in contrast to other materials which may take hours to fully disperse in constantly agitated water.

In particular embodiments, the backing material may be evaluated to meet flushability criteria in accordance with industry guidelines detailed by industry groups such as the European Disposables and Nonwovens Association (EDANA), the Association of the Nonwoven Fabrics Industry (INDA), and the International Water Services Flushability Group (IWSFG). Tests include, but are not limited to, testing the disintegration of the material via the slosh box method detailed in the IWSFG guidelines (IWSFG PAS 1: 2018—Criteria for Recognition as a Flushable Product). Alternatively, testing may include the Toilet and Drainline Clearance Test outlined in INDA/EDANA 2013, FG501. Evaluation of flushability may be altered as guidelines are updated. In some cases, the components used to produce the backing material have been independently determined to comply with flushability guidelines discussed above. In some embodiments, the backing material may not be sufficiently flushable or access to plumbing is limited, and the backing material can instead be discarded in a waste bin.

In addition to the backing material, the collection strip may also comprise one or more materials and be arranged in one or more configurations. In certain embodiments, the collection strip comprises more than one (1) material layer. In other embodiments, the collection strip may comprise only 1 material layer. In still other embodiments the collection strip comprises 1, 2, 3, 4, 5, 6, 7, 8, 9 or 10 layers.

In some embodiments, the collection strip may comprise biologically non-reactive polymers, including but not limited to vinyl, polypropylene(PP), polyethylene(PE), and polyethyleneterephthalate (PET). In some embodiments, the surface of the collection strip may comprise a spunlace nonwoven fiber comprising a synthetic polymer such as those described above.

In some embodiments, the surface of the collection may be textured from the fibers described above to allow for efficient deposition of the sample onto the collection strip. Certain glossy or hydrophobic surfaces may limit the transfer of the sample to the collection strip.

In exemplary embodiments, specific materials should be avoided for the development of the collection strip. For example, fibers or materials derived from plant or animal sources such as cotton, cellulose, papers, wool, silk, and similar materials are to be avoided. Such materials can introduce extraneous DNA that was not present in the sample being collected. Additionally, plant/animal derived fibers, including cellulose, inhibit the recovery of nucleic acids thereby introducing bias in downstream nucleic acid analysis. Additional materials to be avoided for the collection strip are materials that may dissolve in a storage reagent (e.g. DNA/RNA Shield™ available from Zymo Research® in certain embodiments), or introduce impurities/inhibitors that would have a negative impact on downstream analysis of the nucleic acids. These materials include water soluble materials, such as those described for the backing material, or other materials found to degrade in the storage reagent.

In some embodiments, the collection strip may be adhered to the backing material by an acrylic or silicone based adhesive. In some embodiments, the adhesive used to adhere the collection strip to the backing material will be a low tack, removeable adhesive. In such embodiments, the adhesive may include, but is not limited to, a 180 degree peel strength on stainless steel in the range of 3-50 oz/inch.

In some embodiments, the collection strip will include a non-adhesive tab to allow for convenient removal from the backing material. In other embodiments, the adhesive may be incorporated into the backing material such that there is no adhesive on the collection strip.

In current embodiments, the collection strip and the backing material may be manufactured separately. In other embodiments, the collection strip and the backing material may be layered and cut in a single manufacturing process.

### Example

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

The data in the example further shows that the collection strip did not introduce inhibitory compounds into the storage reagent (DNA/RNA Shield™). Next generation sequencing is highly sensitive to certain compounds, and can be inhibited at various steps (library preparation, sequencing, etc.) which would prevent analysis of the sample.

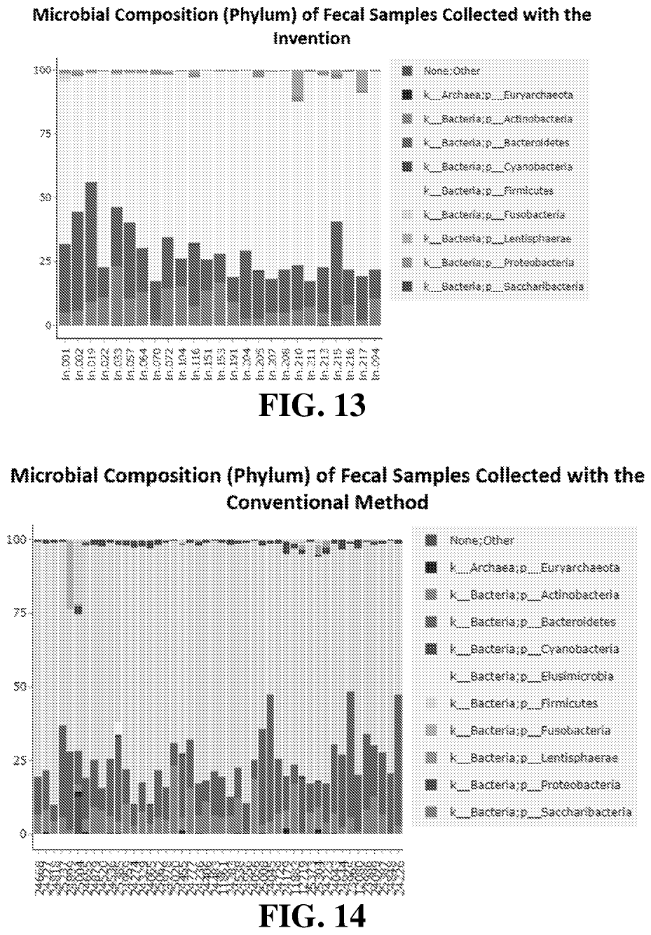

Referring now to FIGS. 13-17, fecal samples were collected from various donors using either an embodiment of the present disclosure, or the conventional method: a small scoop which is used to acquire a consistent quantity from solid stool.

While fecal samples were obtained in the data collected in FIGS. 13-17, exemplary embodiments of the present disclosure can be used on any sample type with non-biased representative results (e.g. skin, blood, saliva, tissue, urine, etc.). A sample for use according to embodiments of the present disclosure may be any sample that comprises or potentially comprises a nucleic acid. For example, the sample may comprise genomic DNA, plasmid DNA or RNA. A sample can be obtained from a variety of sources such as from an animal subject, a plant or from a cell line or tissue bank. A sample may be a fresh sample or a frozen or desiccated sample. For example, a sample from an animal may be a blood sample, a urine sample, a fecal sample, a tissue sample (e.g., a biopsy), a saliva sample, or a hair sample.

After sample collection, the samples were processed and analyzed with the ZymoBIOMICS™ Service—Targeted Metagenomic Sequencing (Zymo Research, Irvine, Calif.).

DNA Extraction

DNA extraction was performed using one of three different DNA extraction kits, depending on the sample type and sample volume. In most cases, the ZymoBIOMICS™ DNA Miniprep Kit (Zymo Research, Irvine, Calif.) was used. For low biomass samples, such as skin swabs, the ZymoBIOMICS™ DNA Microprep Kit (Zymo Research, Irvine, Calif.) was used as it permits for a lower elution volume, resulting in more concentrated DNA samples. For a large sample volume, the ZymoBIOMICS™-96 MagBead DNA Kit (Zymo Research, Irvine, Calif.) was used to extract DNA using an automated platform.

16S Library Preparation

Bacterial 16S ribosomal RNA gene targeted sequencing was performed using the Quick-16S™ NGS Library Preparation Kit (Zymo Research, Irvine, Calif.). The bacterial 16S primers used amplified the V1-V2 or V3-V4 region of the 16S rRNA gene. These primers have been custom-designed by Zymo Research to provide the best coverage of the 16S gene while maintaining high sensitivity. The sequencing library was prepared using an innovative library preparation process in which PCR reactions were performed in real-time PCR machines to control cycles and therefore prevent PCR chimera formation. The final PCR products are quantified with qPCR fluorescence readings and pooled together based on equal molarity. The final pooled library was cleaned up with Select-a-Size DNA Clean & Concentrator™ (Zymo Research, Irvine, Calif.), then quantified with TapeStation® and Qubit®.

Sequencing

The final library was sequenced on Illumina® MiSeg™ with a v3 reagent kit (600 cycles). The sequencing was performed with >10% PhiX spike-in.

Bioinformatics Analysis

Amplicon sequences were inferred from raw reads using the Dada2 pipeline (Callahan et al, 2016). Chimeric sequences were also removed with the Dada2 pipeline. Taxonomy assignment, composition barcharts, alpha-diversity and beta-diversity analyses were performed with Qiime v.1.9.1 (Caporaso et al., 2010). Taxa that have an abundance significantly different among groups were identified by LEfSe (Segata et al., 2011) with default settings if applicable. Other analyses were performed with in-house scripts.

FIG. 13 displays the microbial composition (phylum) of 25 fecal samples, which were collected using an embodiment of the invention described in this application. Samples were analyzed via 16S sequencing.

FIG. 14 displays the microbial composition (phylum) of fecal samples, which were collected using the conventional method of fecal collection. Samples were analyzed via 16S sequencing.

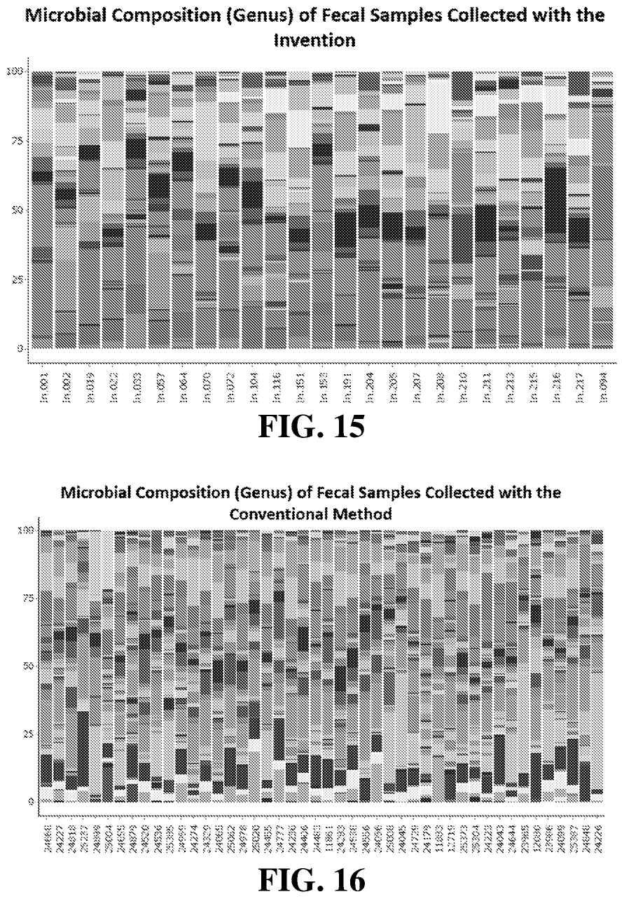

FIG. 15 displays the microbial composition (genus) of 25 fecal samples, which were collected using an embodiment of the invention described in this application. Samples were analyzed via 16S sequencing.

FIG. 16 displays The microbial composition (genus) of fecal samples, which were collected using the conventional method of fecal collection. Samples were analyzed via 16S sequencing.

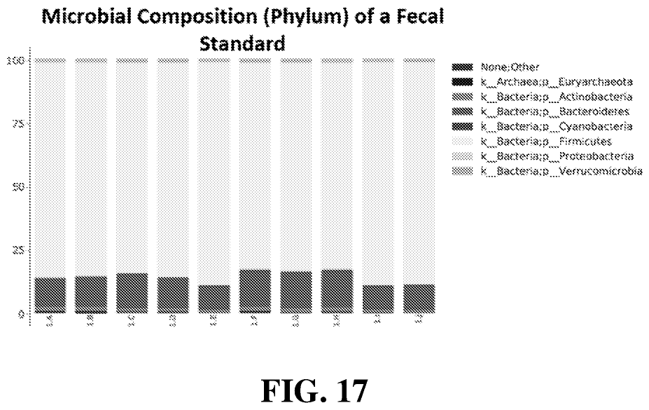

FIG. 17 displays the microbial composition (phylum) of 10 replicates of the same fecal standard. Samples were analyzed via 16S sequencing.

The data presented in FIGS. 13-17 demonstrate that embodiments of the present disclosure were successful in collecting fecal samples for microbiomic analysis when compared to the conventional method.

In FIGS. 13 and 14, it is shown that both methods of fecal collection yield similar distributions of bacterial phyla. In comparison to a pre-defined fecal standard (FIG. 17), the Firmicutes, Bacteroidetes, and the Actinobacteria phyla have the largest representation.

Similarly, in FIGS. 15 and 16, similar distributions of bacteria at the genus level are observed.

Accordingly, embodiments of the invention described in this disclosure can collect fecal samples for downstream analysis of the microbiome in a reliable manner, without a loss of microbial biodiversity or bias towards particular microbes when compared to the conventional ‘scoop’ method.

Microbiome Transport Media (Reagents)

In exemplary embodiments it is important the strip does not interact with sample (fecal matter, or other) transport media to release inhibitors of downstream analysis including but not limited to library preparation and next generation sequencing applications (e.g., DNA/RNA Shield™ and like reagents). In certain embodiments, the sample transport reagents may be a substantially aqueous solution comprising one or more chaotropic salts and, optionally, an alcohol. For example, an embodiment may comprise a chaotropic salt (e.g., guanidinium thiocyanate, guanidinium hydrochloride, sodium iodide, sodium perchlorate, urea or thiourea) or a mixture of chaotropic salts and other chemicals (e.g., salts, sugars, and detergents) and may, or may not, comprise an alcohol. Examples of commercially available sample transport reagents include but are not limited to: RNAlater™ (Ambion™, US; Thermo Fisher Scientific, US), 95% ethanol (VWR International, US), PrimeStore® MTM (Longhorn Vaccines and Diagnostics, US), Stratec (Stratec Molecular GmbH, Germany), OMNIgene®-Gut (DNA 120 Genotek, Canada), Norgen (Norgen Biotek Corp., Canada), DNA/RNA Shield™ (Zymo Research Corp., US), Cary Blair Transport Medium (Remel, US), and

Some sample transport reagents are also amenable to stabilization and purification of nucleic acids (e.g. RNA/DNA) as well as inactivation of microbial pathogens (e.g. viruses, bacteria, and fungi).

### II. Samples

Embodiments of the present disclosure may be used to collect various types of samples. In certain aspects, a biological sample may comprise a cell, milk, blood, serum, plasma, ascites, cyst fluid, pleural fluid, peritoneal fluid, cerebral spinal fluid, wound seepage, semen, vaginal secretions, sweat, tears, urine, feces, saliva, sputum, virus, tissue, plants, or combinations thereof. In certain aspects, the sample is from a mammal, such as human subject.

### III. Downstream Processing

Embodiments of the present disclosure may be used to collect a sample that is subjected to various kinds of down-stream analyses. In some aspects nucleic acids, such as DNA and/or RNA can be isolated from a collected sample. Isolated nucleic acid may be hybridized to a solid support for detection. Isolated nucleic acid may be detected by PCR, qPCR, RT-PCR and/or qRT-PCR. Likewise, nucleic acids may be hybridized to a probe and amplified to aid in detection. Amplified nucleic acids may be detected by PCR, qPCR, Southern blot, northern blot, and/or by sequencing. In some aspects, RNA (e.g., miRNA) may also be ligated to at least one oligonucleotide, reverse transcribed and amplified Amplified RNA products may be detected by qPCR. In certain aspects, nucleic acids may be detected and quantified by hybridization to a microarray. Nucleic acids from a sample or their amplified products can also be analyzed by sequencing, such as by Sanger sequencing, pyrosequencing, or next generation sequencing.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Callahan B. J., McMurdie P. J., Rosen M. J., Han A. W., Johnson A. J., Holmes S. P., (2016) DADA2: High resolution sample inference from Illumina amplicon data. Nat Methods 13(7):581-3.
Caporaso, J. G., Kuczynski, J., Stombaugh, J., Bittinger, K., Bushman, F. D., Costello, E. K. et al. (2010) QIIME allows analysis of high-throughput community sequencing data. Nat Methods 7: 335-336.
Segata, N., Izard, J., Waldron, L., Gevers, D., Miropolsky, L., Garrett, W. S., and Huttenhower, C. (2011) Metagenomic biomarker discovery and explanation. Genome Biol 12: R60.
Plauzolles A., Toumil E., Goutorbel B., Bonnet M., Pénarandal G., Bidaut G., Chiche L., Allardet-Servent J., Retornaz F., Halfon P. Human stool preservation impacts taxonomic profiles in 16S 1 rRNA gene-based metagenomics studies; DOI: 10.21203/rs.3.rs-87437/v1

What is claimed is:

1. A sample collection pad comprising: (a) a sample collection strip; and (b) a backing material having a larger surface area than the sample collecting strip and which is water soluble, said backing material comprising at least two layers, (i) a paper layer and (ii) a tissue layer, wherein the tissue layer is positioned between the paper layer and the sample collection strip.

2. The pad of claim 1, wherein the tissue layer comprises a material that has a higher porosity than the paper layer.

3. The pad of claim 1, wherein the tissue layer has a soft tactile surface.

4. The pad of claim 1, wherein the backing material comprises a starch.

5. The pad of claim 1, wherein the tissue and paper layers are bound together by a water soluble adhesive.

6. The pad of claim 1, wherein the tissue or paper layers comprise a plant-based nonwoven material.

7. The pad of claim 1, wherein the tissue and paper layer conform with the European Disposables and Nonwovens Association (EDANA) and with the Association of the Nonwoven Fabrics Industry (INDA) guidelines for flushability.

8. The pad of claim 1, wherein the tissue layer completely dissolves in water in less than 10 minutes.

9. The pad of claim 1, wherein the sample collection strip comprises a water insoluble material.

10. The pad of claim 1, wherein the sample collection strip comprises a porous or fibrous material.

11. The pad of claim 1, wherein the sample collection strip comprises vinyl, polyethylene(PP), polypropylene(PE), or polyethyleneterephthalate(PET).

12. The pad of claim 1, wherein the sample collection strip comprises a mater that is essentially non-reactive with biological materials.

13. The pad of claim 1, wherein the sample collection strip comprises more than one material layer.

14. The pad of claim 1, wherein the collection pad is sterile.

15. The pad of claim 1, wherein the collection pad is free from nucleic acid material.

16. The pad of claim 1, wherein the collection strip comprises a viral, or microbial, or parasite, or DNA or RNA standard.

17. The pad of claim 1, wherein the sample collection strip comprises an adhesive between the strip and the tissue layer.

18. The pad adhesive of claim 17, wherein the sample collection strip utilizes an acrylic or silicone-based adhesive.

19. The pad of claim 1, wherein the sample collection strip comprises a biological sample.

20. The pad of claim 1, wherein the biological sample comprises material from a wound, a saliva sample, a fecal sample, a urine sample, a mucous sample or a vaginal sample.

21. The pad of claim 1, wherein the biological sample is a fecal sample.

22. The pad of claim 1, wherein the pad is sealed in a packaging slip or pouch.

23. The pad of claim 22, wherein the packaging slip is air-tight.

24. The pad of claim 22, wherein the packaging slip is vacuum sealed.

25. The pad of claim 22, wherein the packaging slip has a sterile interior.

26. The pad of claim 1 wherein the backing material is 1, 2, 3, 4, 5, 6, 7, 8, 9 or 10 layers.

27. A kit comprising a pad accordingly to any one of claims 1-25 and sample storage container.

28. The kit of claim 27, wherein the sample storage container is resealable tube.

29. The kit of claim 27, wherein the sample storage container is sterile.

30. The kit of claim 27, wherein the sample storage container is free of nucleic acid material.

31. The kit of claim 27, wherein the sample storage container comprises a viral, or microbial, or parasite, or DNA or RNA standard.

32. The kit of claim 27, wherein the sample storage container comprises a stabilization reagent.

33. The kit of claim 32, wherein the stabilization reagent comprises an organic solvent or a chaotropic agent.

34. The kit of claim 32, wherein the stabilization reagent comprises guanidinium thiocyanate.

35. The kit of claim 27, further comprising instruction, cleaning pads, sterile gloves, secondary containers or envelopes, and/or labeling materials.

36. A method of collecting a biological sample comprising:
(a) obtaining a pad according to any one of claims 1-25; and
(b) contacting the sample collection strip of the pad to biological material thereby collecting a biological sample.

37. The method of claim 36, further comprising separating the sample collection strip from the backing material.

38. The method of claim 36, further comprising transferring the sample collection strip comprising the biological sample to a sample storage container.

39. The method of claim 38, further comprising labeling the sample storage container.

40. The method of claim 38, further comprising providing the sample storage container to a third party.

41. The method of claim 38, further comprising placing the sample storage container into a secondary container to prevent leakage during transport or storage.

42. The method of claim 38, further comprising mailing the sample storage container.

43. The method of claim 38, wherein the sample storage container is resealable tube.

44. The method of claim 38, wherein the sample storage container is sterile.

45. The method of claim 38, wherein the sample storage container is free of nucleic acid material.

46. The method of claim 38, wherein the sample storage container comprises a viral, or microbial, or parasite, or DNA or RNA standard.

47. The method of claim 38, wherein the sample storage container comprises a stabilization reagent.

48. The method of claim 47, wherein the stabilization reagent comprises an organic solvent or a chaotropic agent.

49. The method of claim 34, wherein the stabilization reagent comprises guanidinium thiocyanate.

50. The method of claim 36, further comprising disposing of the backing material.

51. The method of claim 50, wherein backing material is discarded in a toilet.

52. The method of claim 36, wherein the biological sample comprises material from a wound, a saliva sample, a fecal sample, a urine sample, a mucous sample or a vaginal sample.

53. The method of claim 52, wherein the biological sample is a fecal sample.

54. The method of claim 38, further comprising isolating biological material from the sample collection strip.

55. The method of claim 54, further comprising isolating DNA and/or RNA from sample collection strip.

56. The method of claim 38, further comprising analyzing biological material from the sample collection strip.

57. The method of claim 56, further comprising analyzing DNA and/or RNA from the sample collection strip.

58. The method of claim 57, wherein analyzing comprises sequencing, hybridization and/or PCR.

59. The method of claim 56, further comprising preparing a report with the results of the analysis.

60. The method of claim 56, wherein the analyzing comprises identifying constituent microbial contents in the sample.

61. The method of claim 60, comprising identifying viruses, bacteria and/or parasites present in the sample.

62. The method of claim 60, comprising quantifying the microbial contents of the sample.
